(19) United States
(12) Patent Application Publication
(10) Pub. No.: US 2025/0322940 A1
(43) Pub. Date: Oct. 16, 2025

(54) SYSTEM AND METHOD FOR TRACKING THE LOCATION OF A PATIENT, OBJECT OR MEDICAL PERSONNEL WITHIN A HEALTHCARE ENVIRONMENT

(71) Applicant: Adam Ross, Augusta, GA (US)

(72) Inventor: Adam Ross, Augusta, GA (US)

(21) Appl. No.: 19/022,703

(22) Filed: Jan. 15, 2025

Related U.S. Application Data
(63) Continuation-in-part of application No. 17/375,520, filed on Jul. 14, 2021, now abandoned.
(60) Provisional application No. 63/051,597, filed on Jul. 14, 2020.

(51) Int. Cl. G16H 40/20; G16H 10/60; G16H 40/67

(52) U.S. Cl. CPC G16H 40/20; G16H 10/60; G16H 40/67

(57) ABSTRACT

A system for scheduling a patient for a medical procedure in a healthcare facility, the system comprising: an electronic medical record specific to a patient, wherein the electronic medical record comprises a diagnosis code and at least one health history code; a database of anticipated procedural times for a diagnosis code; and a processor for: (i) analyzing an electronic medical record for a patient for a diagnosis code; (ii) determining a medical procedure to be performed on the patient; (iii) using the database of anticipated procedural times to determine the anticipated procedural time for the medical procedure to be performed on the patient; (iv) analyzing the electronic medical record for the patient for a health history code of the patient; (v) adjusting the anticipated procedural time for the medical procedure to be performed on the patient to a modified procedural time based on the health history code of the patient; and (vi) scheduling space in the healthcare facility for the medical procedure based on the modified procedural time.

### REFERENCE TO PENDING PRIOR PATENT APPLICATIONS

This patent application is a continuation-in-part of pending prior U.S. patent application Ser. No. 17/375,520, filed Jul. 14, 2021 by Adam Ross for SMART/INTEGRATED SYSTEM FOR TRACKING THE LOCATION OF A PATIENT WITHIN A HEALTHCARE ENVIRONMENT (Attorney's Docket No. ROSS-4), which patent application, in turn, claims benefit of prior U.S. Provisional Patent Application Ser. No. 63/051, 597, filed Jul. 14, 2020 by Adam Ross for SMART/INTEGRATED INDOOR POSITIONING SYSTEM (IPS) WITHIN HEALTHCARE ENVIRONMENTS AND OTHER ENVIRONMENTS (Attorney's Docket No. ROSS-4 PROV).

The two (2) above-identified patent applications are hereby incorporated herein by reference.

### FIELD OF THE INVENTION

This invention relates generally to positioning systems for determining the position of a person or object, and more particularly to indoor positioning systems for determining the position of a person or object.

### BACKGROUND OF THE INVENTION

“Where's the patient?”

This phrase is likely said many times daily in every hospital in America.

Patients do not stay in their rooms. Patients are constantly being moved about for diagnostic tests and surgeries/interventions during a hospitalization. By way of example but not limitation, a typical appendicitis admission may look something like this:

ER=>CT Scan=>ER=>Inpatient room=>Pre-op=>OR=>PACU=>Inpatient room

where ER=emergency room, CT Scan=radiology, Inpatient room=patient's room after admission, Pre-Op=pre-operative unit, OR=operating room, and PACU=post-anesthesia care unit.

All within the first 12 hours.

Although Electronic Health Records (sometimes also referred to as Electronic Medical Records, or EMRs) can be updated to show a patient's current location, this is almost universally done manually.

For all hospital staff, a patient's current location is usually shared via word of mouth despite its obvious drawbacks. Many patient locations are still documented on paper, e.g., PACU patient logs. These temporary paper notes of patient location are the “vestigial organs” of a hospital.

### SUMMARY OF THE INVENTION

The present invention comprises the provision and use of a novel smart/integrated system for tracking the location of a patient within a healthcare environment (e.g., a hospital or other healthcare facility), and/or for tracking the location of a medical chart within a healthcare environment, and/or for tracking the location of a medical device within a healthcare environment, and/or for tracking the location of medical personnel (“staff”) within a healthcare environment, and/or for tracking the location of a person within an area, and/or for tracking the location of an object within an area, and/or for tracking the location of an animal within an area (e.g., a zoo), etc.

The data related to the location of a patient, object and/or staff can then be used to enhance scheduling in the healthcare environment, whereby to reduce a patient's length of stay and increase the number of patients that can receive medical care within a healthcare environment.

In one form of the invention, there is provided a system for tracking the location of a patient in a healthcare environment, wherein the healthcare environment comprises an electronic medical record for the patient, wherein the system comprises:
an indoor positioning system (IPS) comprising:
   a plurality of wireless networking beacons for detecting the presence of a wireless tracker module;
   a wireless tracker module configured to move with the patient; and
   a processor in communication with the plurality of wireless networking beacons, wherein the processor is configured to (i) determine the location of the wireless tracker module within the healthcare environment, whereby to determine the location of the patient in the healthcare environment, and (ii) store the location of the patient in the healthcare environment in the electronic medical record for the patient.

In another form of the invention, there is provided a method for tracking the location of a patient in a healthcare environment, wherein the healthcare environment comprises an electronic medical record for the patient, wherein the method comprises:
providing an indoor positioning system (IPS) comprising:
   a plurality of wireless networking beacons for detecting the presence of a wireless tracker module;
   a wireless tracker module configured to move with the patient; and
   a processor in communication with the plurality of wireless networking beacons, wherein the processor is configured to (i) determine the location of the wireless tracker module within the healthcare environment, whereby to determine the location of the patient in the healthcare environment, and (ii) store the location of the patient in the healthcare environment in the electronic medical record for the patient;
placing the wireless tracker module on or near the patient; and
accessing the electronic medical record for the patient to determine the location of the patient in the healthcare environment.

In another form of the invention, there is provided a system for tracking the location of at least one from the group consisting of a person, an object and an animal within an area, wherein the system comprises:
a positioning system comprising:
   a plurality of wireless networking beacons for detecting the presence of a wireless tracker module;
   a wireless tracker module configured to move with the at least one from the group consisting of a person, an object and an animal; and
   a processor in communication with the plurality of wireless networking beacons, wherein the processor is configured to (i) determine the location of the wireless tracker module within the area, whereby to determine the location of the at least one from the group consisting of a person, an object and an animal in the area, and (ii) store the location of the at least one from the group consisting of a person, an object and an animal in the area.

In another form of the invention, there is provided a method for tracking the location of at least one from the group consisting of a person, an object and an animal within an area, the method comprising:
providing a positioning system comprising:
   a plurality of wireless networking beacons for detecting the presence of a wireless tracker module;
   a wireless tracker module configured to move with the at least one from the group consisting of a person, an object and an animal; and
   a processor in communication with the plurality of wireless networking beacons, wherein the processor is configured to (i) determine the location of the wireless tracker module within the area, whereby to determine the location of the at least one from the group consisting of a person, an object and an animal in the area, and (ii) store the location of the at least one from the group consisting of a person, an object and an animal in the area;
placing the wireless tracker module on or near the at least one from the group consisting of a person, an object and an animal; and
accessing the location of the at least one from the group consisting of a person, an object and an animal in the area.

In another form of the invention, there is provided a system for tracking the location of a patient in a healthcare environment in order to improve administrative metrics of the healthcare environment, the system comprising:
an electronic medical record for a patient;
a positioning system comprising:
   a wireless tracker module configured to move with the patient, wherein the wireless tracker module comprises an accelerometer;
   a plurality of wireless networking beacons for detecting the presence of the wireless tracker module, wherein the plurality of wireless networking beacons comprise fixed wireless networking beacons and movable wireless networking beacons; and
   a processor in communication with the plurality of wireless networking beacons, wherein when the accelerometer detects movement of the wireless tracker module, the processor is configured to wait until movement of the wireless tracker module has stopped and (i) determine the time and location of the wireless tracker module within the healthcare environment, whereby to determine the location of the patient in the healthcare environment at a specific time, (ii) store the time and location data of the patient in the healthcare environment in the electronic medical record for the patient, whereby to continuously update the electronic medical record for the patient, (iii) aggregate the time and location data of the wireless tracker module within the healthcare environment to determine how long a patient is in a specific location, (iv) analyze the electronic medical record for the patient for a diagnosis code, and (v) analyze the aggregated time and location data and the diagnosis code from the electronic medical record of the patient to track administrative metrics within the healthcare environment.

In another form of the invention, there is provided a method for tracking the location of a patient in a healthcare environment in order to improve administrative metrics of the healthcare environment, wherein the healthcare environment comprises an electronic medical record for the patient, wherein the method comprises:
providing a positioning system comprising:
   a wireless tracker module configured to move with the patient, wherein the wireless tracker module comprises an accelerometer;
   a plurality of wireless networking beacons for detecting the presence of the wireless tracker module, wherein the plurality of wireless networking beacons comprise fixed wireless networking beacons and movable wireless networking beacons; and
   a processor in communication with the plurality of wireless networking beacons, wherein when the accelerometer detects movement of the wireless tracker module, the processor is configured to wait until movement of the wireless tracker module has stopped and (i) determine the time and location of the wireless tracker module within the healthcare environment, whereby to determine the location of the patient in the healthcare environment at a specific time, and (ii) store the time and location data of the patient in the healthcare environment in the electronic medical record for the patient, whereby to continuously update the electronic medical record for the patient, (iii) aggregate the time and location data of the wireless tracker module within the healthcare environment to determine how long a patient is in a specific location, and (iv) analyze the electronic medical record for the patient for a diagnosis code;
placing the wireless tracker module on or near the patient;
determining the time and location of the wireless tracker module in the healthcare environment at a first point in time;
determining the time and location of the wireless tracker module in the healthcare environment at a second point in time;
aggregating the time and location data of the wireless tracker module within the healthcare environment to determine how long a patient is in a specific location;
analyzing the electronic medical record for the patient for a diagnosis code;
analyzing the aggregated time and location data of the patient and the diagnosis code of the patient to track administrative metrics, whereby to improve wait times within the healthcare environment.

In another form of the invention, there is provided a system for scheduling a patient for a medical procedure in a healthcare facility, the system comprising:
an electronic medical record specific to a patient, wherein the electronic medical record comprises a diagnosis code and at least one health history code;
a database of anticipated procedural times for a diagnosis code; and
a processor for:
(i) analyzing an electronic medical record for a patient for a diagnosis code;
(ii) determining a medical procedure to be performed on the patient;
(iii) using the database of anticipated procedural times to determine the anticipated procedural time for the medical procedure to be performed on the patient;
(iv) analyzing the electronic medical record for the patient for a health history code of the patient;
(v) adjusting the anticipated procedural time for the medical procedure to be performed on the patient to a modified procedural time based on the health history code of the patient; and
(vi) scheduling space in the healthcare facility for the medical procedure based on the modified procedural time.

In another form of the invention, there is provided a method for scheduling a patient for a medical procedure in a healthcare facility, wherein the healthcare environment comprises an electronic medical record specific to a patient, and further wherein the electronic medical record comprises a diagnosis code and at least one health history code, wherein the method comprises:
providing a database of anticipated procedural times for a diagnosis code;
analyzing an electronic medical record for a patient for a diagnosis code;
determining a medical procedure to be performed on the patient;
using the database of anticipated procedural times to determine the anticipated procedural time for the medical procedure to be performed on the patient;
analyzing the electronic medical record for the patient for a health history code of the patient;
adjusting the anticipated procedural time for the medical procedure to be performed on the patient to a modified procedural time based on the health history code of the patient; and
scheduling space in the healthcare facility for the medical procedure based on the modified procedural time.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a novel smart/integrated indoor positioning system (IPS) for a healthcare environment;

FIG. 2 is a schematic view showing a patient with a wearable wireless tracker module;

FIG. 3 is a schematic view showing an electronic medical record continuously updated with the location of the patient;

FIG. 4 is a schematic view showing a medical chart with a wireless tracker module;

FIG. 5 is a schematic view showing a medical device with a wireless tracker module; and

FIG. 6 is a schematic view showing medical staff with a wearable wireless tracker module.

### DETAILED DESCRIPTION OF THE PREFERRED EMBODIMENTS

### Smart/Integrated Indoor Positioning System (IPS) In General

The present invention comprises the provision and use of a novel smart/integrated indoor positioning system (IPS) for tracking the location of a patient within a healthcare environment (e.g., a hospital or other healthcare facility), and/or for tracking the location of a medical chart within a healthcare environment, and/or for tracking the location of a medical device within a healthcare environment, and/or for tracking the location of medical personnel (“staff”) within a healthcare environment, and/or for tracking the location of a person within an area, and/or for tracking the location of an object within an area, and/or for tracking the location of an animal within an area (e.g., a zoo), etc.

For the sake of example but not limitation, the present invention will first be discussed in the context of tracking the location of a patient within a healthcare environment (e.g., a hospital or healthcare facility), and/or tracking the location of a medical chart within a healthcare environment, and/or tracking a medical device within a healthcare environment, and/or tracking a medical professional (“staff”) in a healthcare environment. However, it should be appreciated that the present invention may also be applicable to tracking the location of a person within an area and/or to tracking an object within an area and/or to tracking an animal within an area (e.g., a zoo), etc.

The data related to the location of a patient, object and/or staff can then be used to enhance scheduling in the healthcare environment, whereby to reduce a patient's length of stay and increase the number of patients that can receive medical care within a healthcare environment.

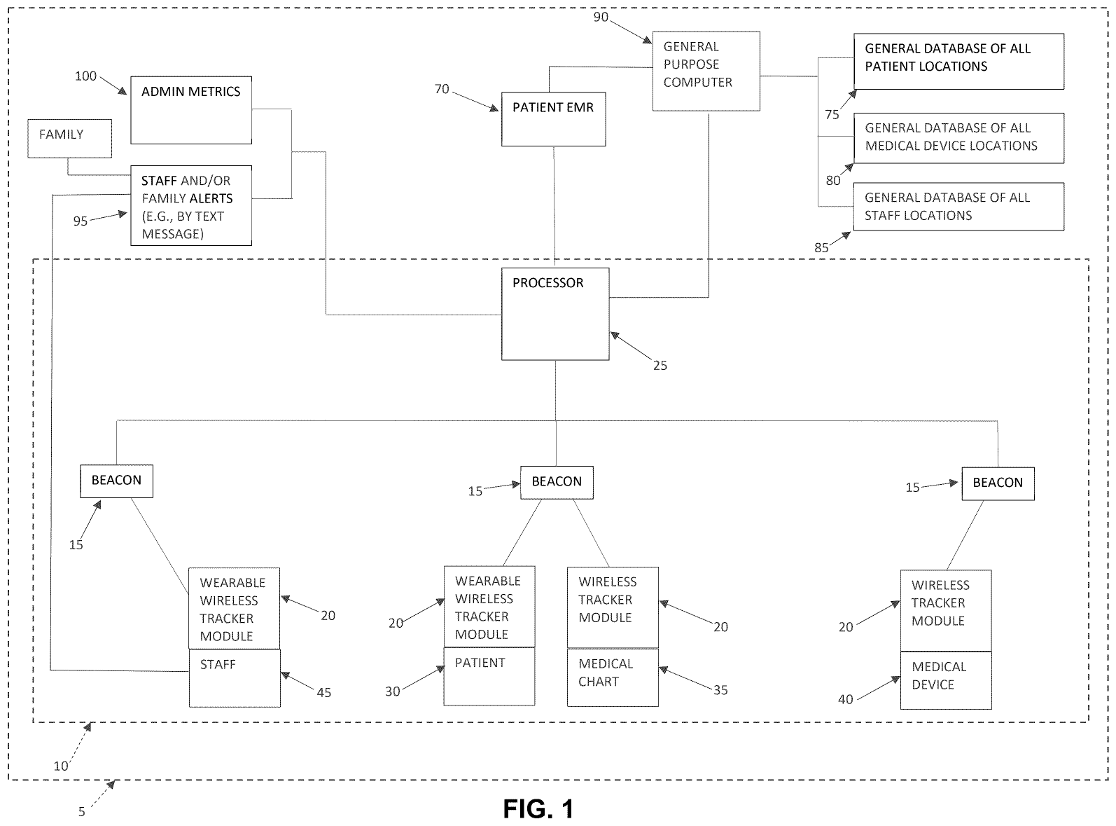

Looking first at FIG. 1, there is shown a novel smart/integrated indoor positioning system (IPS) 5 for tracking the location of a patient within a healthcare environment (e.g., a hospital or other healthcare facility), and/or for tracking the location of a medical chart within a healthcare environment, and/or for tracking the location of a medical device within a healthcare environment, and/or for tracking the location of medical personnel (“staff”) within a healthcare environment. System 5 generally comprises an indoor positioning system (IPS) 10 which comprises a plurality of beacons 15 for identifying the location of wireless tracker modules 20 and reporting those locations to a processor 25.

More particularly, whenever a particular wireless tracker module 20 is within the range of a particular beacon 15, that beacon 15 reports this fact to processor 25 (e.g., such as by sending a message to processor 25 reporting that “Beacon 15i has detected the presence of a wireless tracker module 20j within its operating zone”). Note that each beacon 15 and each wireless tracker module 20 has a unique identifier. In this way, processor 25 can keep track of the current location of any wireless tracker module 20 which is within the range of any beacon 15.

Furthermore, processor 25 is configured to correlate a particular wireless tracker module 20 with a particular patient, or medical chart, or medical device, or medical professional, etc. (e.g., via a look-up table containing the appropriate data, such as “Wireless Tracker Module 20q is assigned to patient John Smith; Wireless Tracker Module 20r is assigned to patient Jane Doe; Wireless Tracker Module 20s is assigned to the medical chart for patient Mary Gray”, etc.).

Additionally, processor 25 is configured to correlate a particular beacon 15 with a particular geographic location in the healthcare environment (e.g., via a look-up table containing the appropriate information, such as “Beacon 15x is located in the Emergency Room; Beacon 15y is located in Radiology; Beacon 15z is located in Patient Room 703”, etc.).

In this way, the location of any patient, medical chart, medical device, healthcare professional, etc. who/which has been provided with a unique wireless tracker module 20 can be determined so long as that unique wireless tracker module 20 is within the range of a beacon 15, and the location of that patient, medical chart, medical device, healthcare professional, etc. can be identified in both (i) the context of a particular beacon 15, and/or (ii) the context of a particular geographic location in the healthcare environment, e.g., Emergency Room, Radiology, Patient Room 703, etc.

Note that, whenever a beacon 15 reports the presence of a wireless tracker module 20, processor 25 can be configured to “timestamp” that event (i.e., record the time at which a beacon 15 reports the presence of a wireless tracker module 20).

Indoor positioning system (IPS) 10 may use many different modalities to determine the position of wireless tracker modules 20 including Wi-Fi, Bluetooth, ultra-wideband (UWB), radiofrequency (RF) devices, etc., and any combination thereof. Beacons 15 and wireless tracker modules 20 are consistent with the modality used (e.g., where indoor position system (IPS) 10 uses Wi-Fi technology, beacons 15 and wireless tracker modules 20 are Wi-Fi units; where indoor positioning system (IPS) uses Bluetooth technology, beacons 15 and wireless tracker modules 20 are Bluetooth units; where indoor positioning system (IPS) uses radiofrequency (RF) devices, beacons 15 and wireless tracker modules 20 are radiofrequency (RF) units, etc.).

Note that as healthcare environments have become more integrated with wireless internet, the presence of Wi-Fi has become almost universal within healthcare environments. As a result, Wi-Fi is a preferred choice for an indoor positioning system within a healthcare environment.

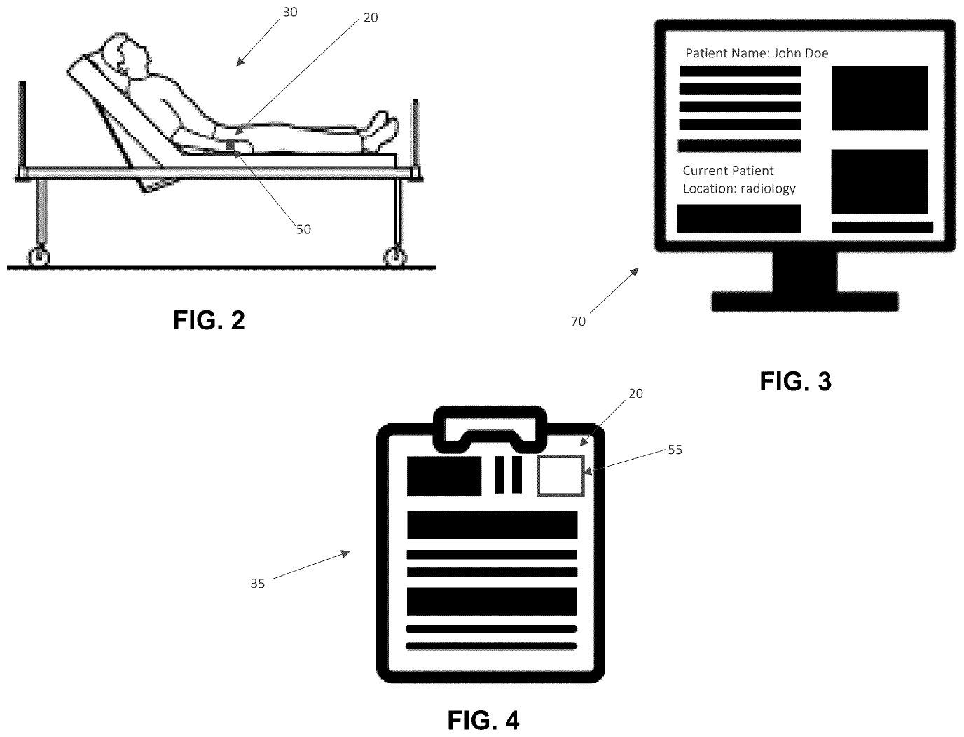

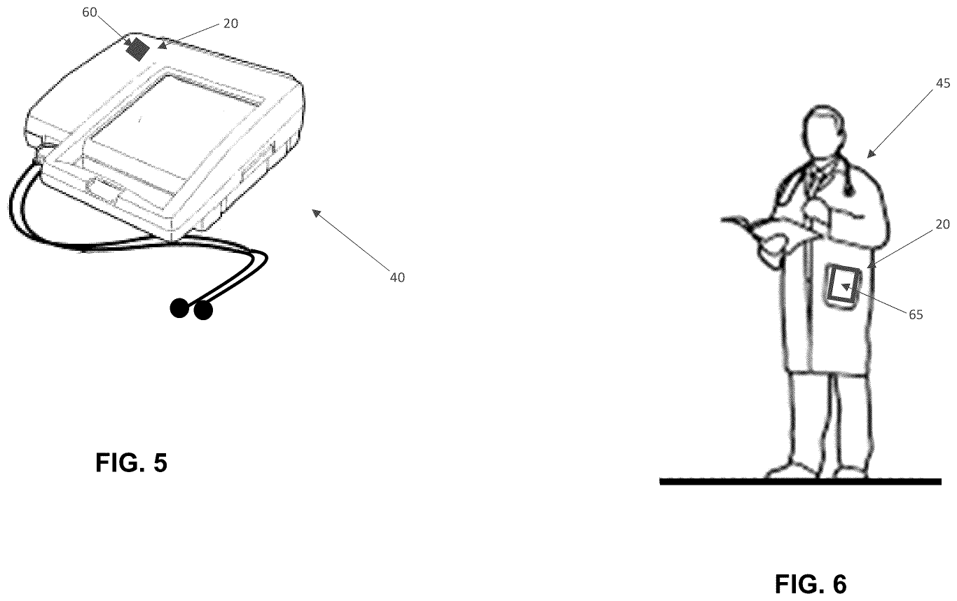

Wireless tracker modules 20 may be used to track the location of patients 30, medical charts 35, medical devices 40, or medical professionals 45. Wireless tracker modules 20 are provided in a form consistent with the person or object which is to be tracked. By way of example but not limitation, where a patient 30 is to be tracked, wireless tracker module 20 may comprise a bracelet 50 (see FIG. 2); where a medical chart 35 is to be tracked, wireless tracker module 20 may comprise an electronic tag 55 adhesively attached to the medical chart (see FIG. 4); where a medical device 40 is to be tracked, wireless tracker module 20 may comprise an electronic tag 60 adhesively attached to the medical device (see FIG. 5); where a medical professional 45 is to be tracked, wireless tracker module 20 may comprise a badge 65 (see FIG. 6).

Processor 25 (which may comprise an appropriately-programmed general purpose computer) is configured to store the locations of wireless tracker modules 20 for patients 30, and to store the locations of wireless tracker modules 20 for medical charts 35, into patient EMRs 70, and/or into a general database of all patient locations 75; and to store the locations of wireless tracker modules 20 for medical devices 40 into a general database of medical device locations 80; and to store the locations of wireless tracker modules 20 for medical personnel 45 into a general database of all staff locations 85, etc. Note that as processor 25 stores the locations of wireless tracker modules 20, processor 25 preferably also stores the time at which these locations are determined, whereby to “timestamp” that event.

As a result, when hospital personnel need to determine the location of a patient, the medical personnel may use a general purpose computer 90 to access a patient EMR 70 to learn the location of that patient. Alternatively, when medical personnel need to determine the location of a patient, the medical personnel may use general purpose computer 90 to query general database of all patient locations 75 to learn the location of that patient.

Similarly, when medical personnel need to determine the location of a medical device 40, the medical personnel may use general purpose computer 90 to query general database of all medical device locations 80 to learn the location of that medical device.

Further, when medical personnel need to determine the location of other medical personnel, the medical personnel can use general purpose computer 90 to query general database of all medical personnel locations 85 to learn the location of that medical personnel.

### Significant Aspects of the Present Invention

In accordance with the present invention, and looking now at FIGS. 1 and 2, a wearable wireless tracker module 20, such as a Wi-Fi module, is used to individually tag a patient 30 and track their movements within a hospital environment. In this form of the invention, the wearable wireless tracker module 20 may comprise a “digital name bracelet” that can be “seen” by a wireless network (such as a Wi-Fi network) using beacons 15 to track the location of the wearable wireless tracker module 20, so that the current location of the wearable wireless tracker module 20 (and hence the current location of the patient 30) can be determined by the hospital's wireless network.

Ideally, the wearable wireless tracker module 20 is physically integrated into the patient's name bracelet or added as an additional wrist band obtained at arrival in the hospital (e.g., bracelet 50). The wearable wireless tracker module 20 may also be attached to the patient's name bracelet.

As the patient is moved from one area of the hospital to another, the location of the patient may be continuously tracked by wearable wireless tracker module 20 and the location of the patient is automatically continuously updated in the electronic medical record (EMR) 70 for that patient (see FIG. 3) or sent to other applications such as existing messaging software. This data, along with timestamps, can be logged into the EMR 70 and/or into an external database (e.g., a general database of all patient locations 75, see FIG. 1).

In one important aspect of the invention, the patient's movement to or from an area can also trigger secondary actions. By way of example but not limitation, the charge nurse can get a text message when a new patient arrives on the unit; and/or a surgeon can receive a message when his patient is in the OR; and/or housekeeping can be notified when a patient is discharged and a room needs to be turned over; and/or family can be notified when a patient gets out of the OR and into the PACU. See alerts 95 in FIG. 1.

The location data can also be aggregatable. For example, an “inpatient” physician could see a live list of all of their patients' locations.

Timestamping location data also allows valuable information to be gathered by the system. By way of example but not limitation, timestamping location data can allow the system to determine how long a given patient spends in the PACU, and/or how long it takes for a given patient to go from “door to balloon time”, and/or how long a given patient is held in the ER, etc. See admin metrics 100 in FIG. 1. Aggregating timestamped location data allows additional valuable information to be gathered by the system, e.g., it would allow things like “average PACU time”, “door to balloon time” and “ER holding times” to be easily tracked for quality improvement. Again, see admin metrics 100 in FIG. 1.

As the patient moves, they can be “passed off” between nurses, e.g., such as by issuing alerts 95 to the appropriate nurses. This will allow more continuity of care during a hospital stay such as when giving medications.

Wearable devices that give notifications have been used for many years in nurseries. They are currently configured to provide a simple binary answer: baby here vs. baby gone. “Code pink” is still used for lost pediatric patients, so such systems obviously are not foolproof. These prior art systems generally use “geofencing” and rarely have a backup. The present invention provides a significant improvement over these prior art “geofencing” systems, since indoor positioning system (IPS) 10 can identify the location of any patient via their wearable wireless tracker module 20 and issue an alert (e.g., an alert 95) if a patient has left an approved location.

The present invention may also be configured to provide relative geofencing, for example between patients, where the novel system could be used to keep certain patients/people/objects away from each other. By way of example but not limitation, in this form of the invention, the indoor positioning system (IPS) 10 can identify the locations of any patients via their wearable wireless tracker modules 20, and identify when certain patients come into close proximity to one another, and then send an alert to medical personnel 45 via an alert 95.

A significant aspect of the present invention is the automatic integration of the patient location information within the Electronic Medical Record (EMR) and allowing for live notifications regarding the location of patients via messaging software or other wired and/or wireless means.

In some cases, it may be desirable to track the location of the patient without using a wearable wireless tracker module (e.g., such as where it may be undesirable to have a wearable electronic device immediately adjacent to the patient's body). In this case, it can be desirable to place a tracker on the patient's physical chart, since the patient's physical chart typically moves with the patient. See FIG. 4.

The system can also be used to tag medical devices (see FIG. 5) as well as medical staff (see FIG. 6). This could help streamline care and determine things like “time at bedside”. Quick tallies of IV poles or beds physically on floor could easily be tracked using the present invention (e.g., via medical device tracking).

A wearable or non-wearable Wi-Fi module (i.e., a wearable or non-wearable wireless tracker module 20 utilizing a Wi-Fi modality) will drain a battery (particularly a small, lightweight battery) at a fast rate. A significant aspect of the present invention addresses this limitation: the wearable or non-wearable Wi-Fi's module's default condition is to be “turned off”. At certain times, e.g., every 3-5 minutes, the wearable or non-wearable Wi-Fi module will turn on to determine its current location (and hence the current location of the wearer or the person or object to which the wearable or non-wearable Wi-Fi module is associated). This feature may sometimes be referred to herein as “periscoping”. The timing could be changed based on typical movement times, e.g., more frequently during the day when the patient is more likely to be moving about, less frequently at night when the patient is more likely to be sleeping, etc. An accelerometer can also be used to detect patient or object or staff movement and to override the usual “turn on” times, e.g., if the accelerometer senses movement, the wearable or non-wearable Wi-Fi module waits until the movement has stopped and then turns on to report the patient's new location.

Bluetooth-based systems generally require hardware installations (i.e., Bluetooth “beacons” 15) in hospitals for the system to function. These Bluetooth beacons 15 are placed as needed and can use triangulation to determine the presence of the wireless Bluetooth tracker module 20 (e.g., using the patient's Bluetooth wrist band 20). However, installing numerous Bluetooth beacons is an expensive proposition in large, multi-level hospitals. In a significant aspect of the present invention, the system instead relies on existing hardwired computers that have Bluetooth capability and taps these Bluetooth-enabled computers to provide the Bluetooth beacons 15 used in the new system. Standalone Bluetooth beacons 15, hardwired or plugged into regular outlets, could be used alone or in conjunction with the Bluetooth beacons 15 within desktop computers. Multiple handheld devices, such as cellphones or tablets, could also be used as Bluetooth beacons 15 to complete a wireless Bluetooth network with wired and wireless beacons 15.

Using existing Bluetooth beacons in existing computers will not give the most optimized performance of the system, however, it eliminates what is likely to be a large barrier to entry (i.e., the cost of setting up a Bluetooth beacon network).

Bluetooth battery life is far superior to Wi-Fi, as well as is Bluetooth's ability to accurately triangulate position. The wearable Bluetooth module 20 may also be configured, in ways well known in the art, to collect and transmit live patient information such as vital signs. The wireless wearable Bluetooth module 20 may also be configured, in ways well known in the art, to take the place of a call bell, i.e., it could provide patient-to-nurse communication via integration of a speaker.

In the foregoing discussion, the present invention is discussed in the context of tracking patients, medical charts, medical devices and/or medical personnel within a healthcare environment (e.g., a hospital or other healthcare facility). However, it should be appreciated that the present invention is also applicable to tracking the location of persons within an area and/or tracking objects within an area and/or tracking animals within an area (e.g., a zoo), etc.

### Exemplary Use of the Present Invention

As a result of the foregoing, novel smart/integrated indoor positioning system (IPS) 5 may be used to track locations of patients, medical charts, medical devices and/or medical personnel within a healthcare environment (e.g., a hospital or other healthcare facility); and/or to track the location of persons within an area; and/or to track objects within an area; and/or to track animals within an area (e.g., a zoo), etc.

In an exemplary use of the present invention, novel smart/integrated indoor positioning system (IPS) 5 may be used to track a patient during a hospital stay. More particularly, and looking now at FIGS. 1-3, when a patient 30 is admitted to the hospital, patient 30 may be fitted with a wearable wireless tracker module 20 in the form of a bracelet 50. Patient 30 may thereafter be placed in a room. The patient's EMR 70 is then automatically updated with the patient's location when bracelet 50 communicates with the nearest beacon 15, which in turn communicates with processor 25, which in turn communicates with the patient's EMR 70. In addition, processor 25 may also store the location of bracelet 50 into a general database of all patient locations 75. Alternatively and/or additionally, the medical chart 35 for that patient may be fitted with a wireless tracker module 20; since the medical chart generally travels with the patient, tracking the location of medical chart 35 via wireless tracker module 20 generally provides the current location of the patient. Again, the wireless tracker module 20 for medical chart 35 may be stored in the patient's EMR 70 and/or the general database of all patient locations 75.

When patient 30 is thereafter moved from their room to radiology for testing, EMR 70 is continuously updated with the current location of the patient as bracelet 50 (and/or the wireless tracker module 20 for medical chart 35) communicates with various beacons 15 (which in turn communicate with processor 25) while the patient moves through the hospital.

Once patient 30 is done in radiology and moving back to their room, or to another area of the hospital for additional testing, EMR 70 is once again continuously updated as bracelet 50 (and/or the wireless tracker module 20 for medical chart 35) communicates with various beacons 15 (which in turn communicate with processor 25) while the patient moves through the hospital. If medical personnel need to know the location of patient 30, the medical personnel may use a general purpose computer 90 to access the patient's EMR 70 to learn the current location of the patient (e.g., radiology). Alternatively, the medical personnel may use general purpose computer 90 to query a general database of all patient locations 75 to learn the location of the patient.

In this way, a patient's current location is always readily available to medical personnel.

It should be appreciated that in the foregoing example, the present invention is discussed in the context of tracking a patient within a hospital, however, the present invention is also applicable to tracking the location medical devices and/or medical personnel within a hospital; and/or to tracking persons within an area and/or to tracking objects within an area and/or to tracking animals within an area (e.g., a zoo), etc.

### Enhanced Scheduling Using Novel System 5

Existing operating room scheduling is essentially analog. More particularly, with existing operating room scheduling, each day's schedule is set by hand with a general order and estimated blocks of time. This “analog scheduling” generally leads to “choke points” in either the operating rooms themselves, the pre-op area (pre-op) or the post-anesthesia care unit area (PACU), each of which areas has a limited number of beds available. By way of example but not limitation, delays in one operating room can lead to delays in the pre-op area by overwhelming the pre-op area with patients waiting to be moved to the operating room. Inefficiencies in scheduling also lead to understaffing in one area and overstaffing in another area. Thus there is a need in the art for a system that provides for patient-specific scheduling, which permits updating of operating room scheduling in real-time, and which facilitates more accurate scheduling.

To that end, in another embodiment of the invention, novel system 5 may be used to provide enhanced scheduling for use of certain spaces (e.g., an operating room, a recovery room, etc.) in a hospital.

More particularly, if desired, system 5 may further comprise a general database of diagnosis codes 105 and a general database of anticipated procedural times 110 keyed to the general database of diagnosis codes 105. With this embodiment of the invention, anticipated procedural times can be assigned to each particular diagnosis code (e.g., based on predicted procedural times that are empirically-derived based on experience performing the procedures represented by the diagnosis codes contained in general database of diagnosis codes 105).

By way of example but not limitation, general database of diagnosis codes 105 may contain a diagnosis code for the procedure sometimes referred to as an “appendectomy”, linked to database of anticipated procedural time 110 which specifies that 1 hour is assigned for that procedure for pre-operative (pre-op) time relative to that diagnosis code, 2 hours of operating room (OR) time relative to that diagnosis code, 2 hours of post anesthesia care unit (PACU) time, etc. As a result, when the diagnosis code for “appendectomy” is present, system 5 would determine that 5 hours of time in total are required for prepping patient 30 for surgery, performing the operation, and providing for recovery from the operation, with the time assigned to different areas of the hospital (e.g., pre-op, OR, PACU, etc.). Thus, system 5 may use the diagnosis code stored in general database of diagnosis codes 105 to determine how much time in each of several different areas of the hospital (e.g., pre-op, OR, PACU, etc.) should be reserved for a particular procedure for a particular patient, thereby facilitating scheduling of those different areas of the hospital.

However, it should also be appreciated that system 5 may utilize software (e.g., AI-enhanced software) to modify anticipated procedural times stored in general database of anticipated procedural times 110 so as to be patient-specific. By way of example but not limitation, it may be found through experience that an appendectomy performed on an obese patient requires significantly more time in the operating room than when the patient is not obese. System 5 is configured to utilize patient-specific information (e.g., a particular patient's co-morbidities such as obesity, history of smoking, and/or anything else that may affect a procedure time) stored in patient EMR 70 in the form of a health history code for a particular patient to modify an anticipated procedural time so as to accommodate a particular patient. Thus, when system 5 determines that patient 30 is to be scheduled for, for example, an appendectomy (e.g., patient 30's EMR 70 includes a diagnosis code for appendectomy as keyed to general database of diagnosis codes 105), system 5 may review EMR 70 and, if a health history code corresponding to a co-morbidity such as obesity is present in EMR 70, system 5 may modify the anticipated procedural time retrieved from general database of anticipated procedural times 110 in a manner which accommodates the needs of patient 30. By way of example but not limitation, in the foregoing example, system 5 may, for example, modify the anticipated OR time from 2 hours to 3.5 hours in order to account for the extra time in surgery required to perform an appendectomy in an obese patient (or extra time required for an elderly patient, a child patient, a generally ill patient with other existing co-morbidities, etc.). Similarly, pre-op and/or PACU time may also be modified as necessary to account for the health history code corresponding to the particular patient's medical condition (presence of co-morbidities, patient age, patient general health, etc.) of patient 30.

To this end, it should be appreciated that system 5 may employ AI-enhanced software to determine patient scheduling, with the result that operating room schedules (and/or pre-op space schedules, PACU space schedules, etc.) may be run in a manner analogous to airports-providing continuously updated times for arrivals of particular patients into particular spaces and departures of particular patients out of particular spaces. By tracking patient locations within the hospital in the manner discussed above, and by calculating anticipated preparation, procedure, and recovery times for known procedures based on patient-specific data contained in patient-specific EMR 70, accurate estimated start times for each individual patient can be predicted and updated continuously by system 5. It will also be appreciated that staff scheduling can be tailored using this data as well (e.g., by tracking locations of badges 65, correlating locations with patients and procedures, and determining when a particular staff member should be in a particular location).

Thus, system 5 provides a tailored schedule for each operating room (and/or pre-op space, PACU space, etc.) based on the average OR times of particular surgeons, anesthesiologists and OR teams combined with a patient's individual diagnoses/EMR 70 so as to allow for more efficient patient flow through the operating rooms/procedure rooms. This, in turn, results in a decrease in patient length of stay and an increase in the number of patients that can receive medical care.

As discussed above, bracelet 50 and/or electronic tag 55 permit real-time physical location tracking of each particular patient's medical chart 35 (which always accompany the patient) and/or the patient 30. The location data relative to each particular patient 30 as they move through the hospital may be combined with a diagnosis code associated with that patient (e.g., stored in EMR 70 and keyed to general database of diagnosis codes 105) and the particular patient's health history code (e.g., a code corresponding to at least one patient-specific condition such as obesity, high-blood pressure, high cholesterol, etc.). The data may be compiled and used to predict OR times given individual needs, allowing for predictable turn-over times for each operating room and, ultimately, the performance of more operations in a given period of time. In other words, by combining the use of (i) diagnosis codes stored in general database of diagnosis codes 105, (ii) the anticipated times to be spent in pre-op/OR/PACU stored in general database of anticipated procedural time 110, and (iii) a health history code stored in EMR 70 specific to each patient 30, patient-specific scheduling can generated. Then, the AI-enhanced software of system 5 is able to create a dynamic, real-time schedule based on tendencies of individual surgeons, OR teams, and the specific needs of a particular patient.

### Modifications

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

In the claims:

1. A system for tracking the location of a patient in a healthcare environment in order to improve administrative metrics of the healthcare environment, the system comprising:
an electronic medical record for a patient;
a positioning system comprising:
a wireless tracker module configured to move with the patient, wherein the wireless tracker module comprises an accelerometer;
a plurality of wireless networking beacons for detecting the presence of the wireless tracker module, wherein the plurality of wireless networking beacons comprise fixed wireless networking beacons and movable wireless networking beacons; and
a processor in communication with the plurality of wireless networking beacons, wherein when the accelerometer detects movement of the wireless tracker module, the processor is configured to wait until movement of the wireless tracker module has stopped and (i) determine the time and location of the wireless tracker module within the healthcare environment, whereby to determine the location of the patient in the healthcare environment at a specific time, (ii) store the time and location data of the patient in the healthcare environment in the electronic medical record for the patient, whereby to continuously update the electronic medical record for the patient, (iii) aggregate the time and location data of the wireless tracker module within the healthcare environment to determine how long a patient is in a specific location, (iv) analyze the electronic medical record for the patient for a diagnosis code, and (v) analyze the aggregated time and location data and the diagnosis code from the electronic medical record of the patient to track administrative metrics within the healthcare environment.

2. The system according to claim 1 wherein the processor is further configured to analyze the aggregated time and location data and the diagnosis code from the electronic medical record of the patient to determine a patient schedule.

3. The system according to claim 2 wherein the system comprises a plurality of electronic medical records, and further wherein the processor is configured to analyze the aggregated time and location data and the diagnosis code from the plurality of electronic medical records to determine a patient schedule for a plurality of patients.

4. The system according to claim 1 wherein the wireless tracker module is secured to the patient so as to move with the patient.

5.-8. (canceled)

9. The system according to claim 1 wherein the wireless tracker module is carried by the patient's physical medical chart so as to move with the patient.

10. The system according to claim 1 wherein the processor is further configured to store the location of the patient in the healthcare environment in a general database of all patient locations.

11. The system according to claim 1 wherein the processor is further configured to provide alerts based on the location of the patient in the healthcare environment.

12.-15. (canceled)

16. The system according to claim 1 wherein the default condition of the wireless tracker module is to be turned off.

17. The system according to claim 16 wherein the wireless tracker module is configured to turn on at set time intervals.

18.-19. (canceled)

20. The system according to claim 1 wherein the wireless tracker module turns on after the accelerometer has stopped detecting movement.

21.-25. (canceled)

26. The system according to claim 1 wherein the wireless tracker module is configured to transmit the patient's vital signs to the processor, and further wherein the processor is further configured to store the patient's vital signs in the electronic medical record for the patient.

27. The system according to claim 1 wherein the wireless tracker module comprises a speaker, and further wherein the wireless tracker module is configured to provide communication between the patient and medical personnel.

28. The system according to claim 1 wherein the system is also configured to track the location of a medical device in the healthcare environment.

29. The system according to claim 1 wherein the system is also configured to track the location of medical personnel in the healthcare environment.

30. A method for tracking the location of a patient in a healthcare environment in order to improve administrative metrics of the healthcare environment, wherein the healthcare environment comprises an electronic medical record for the patient, wherein the method comprises:
providing a positioning system comprising:
a wireless tracker module configured to move with the patient, wherein the wireless tracker module comprises an accelerometer;
a plurality of wireless networking beacons for detecting the presence of the wireless tracker module, wherein the plurality of wireless networking beacons comprise fixed wireless networking beacons and movable wireless networking beacons; and
a processor in communication with the plurality of wireless networking beacons, wherein when the accelerometer detects movement of the wireless tracker module, the processor is configured to wait until movement of the wireless tracker module has stopped and (i) determine the time and location of the wireless tracker module within the healthcare environment, whereby to determine the location of the patient in the healthcare environment at a specific time, and (ii) store the time and location data of the patient in the healthcare environment in the electronic medical record for the patient, whereby to continuously update the electronic medical record for the patient, (iii) aggregate the time and location data of the wireless tracker module within the healthcare environment to determine how long a patient is in a specific location, and (iv) analyze the electronic medical record for the patient for a diagnosis code;
placing the wireless tracker module on or near the patient;
determining the time and location of the wireless tracker module in the healthcare environment at a first point in time;
determining the time and location of the wireless tracker module in the healthcare environment at a second point in time;
aggregating the time and location data of the wireless tracker module within the healthcare environment to determine how long a patient is in a specific location;
analyzing the electronic medical record for the patient for a diagnosis code;
analyzing the aggregated time and location data of the patient and the diagnosis code of the patient to track administrative metrics, whereby to improve wait times within the healthcare environment.

31. A system for scheduling a patient for a medical procedure in a healthcare facility, the system comprising:
an electronic medical record specific to a patient, wherein the electronic medical record comprises a diagnosis code and at least one health history code;
a database of anticipated procedural times for a diagnosis code; and
a processor for:
(i) analyzing an electronic medical record for a patient for a diagnosis code;
(ii) determining a medical procedure to be performed on the patient;
(iii) using the database of anticipated procedural times to determine the anticipated procedural time for the medical procedure to be performed on the patient;
(iv) analyzing the electronic medical record for the patient for a health history code of the patient;
(v) adjusting the anticipated procedural time for the medical procedure to be performed on the patient to a modified procedural time based on the health history code of the patient; and
(vi) scheduling space in the healthcare facility for the medical procedure based on the modified procedural time.

32. A method for scheduling a patient for a medical procedure in a healthcare facility, wherein the healthcare environment comprises an electronic medical record specific to a patient, and further wherein the electronic medical record comprises a diagnosis code and at least one health history code, wherein the method comprises:
providing a database of anticipated procedural times for a diagnosis code;
analyzing an electronic medical record for a patient for a diagnosis code;
determining a medical procedure to be performed on the patient;
using the database of anticipated procedural times to determine the anticipated procedural time for the medical procedure to be performed on the patient;
analyzing the electronic medical record for the patient for a health history code of the patient;
adjusting the anticipated procedural time for the medical procedure to be performed on the patient to a modified procedural time based on the health history code of the patient; and
scheduling space in the healthcare facility for the medical procedure based on the modified procedural time.

33. The system of claim 31 further comprising a database of typical procedural times corresponding to medical professionals associated with performing the medical procedure, and wherein the processor is further configured to schedule space in the healthcare facility for the medical procedure based on the typical procedural time.

34. The system of claim 31 further comprising a wireless tracker module configured to move with at least one from the group consisting of the patient, a medical professional, hospital staff and a medical device to determine the time and location of the wireless tracker module within the healthcare environment.

35. The method of claim 32 further comprising (i) providing a database of typical procedural times corresponding to medical professionals associated with performing the medical procedure, (ii) using the database of typical procedural times corresponding to medical professionals associated with performing the medical procedure to further adjust the modified procedural time, and (iii) scheduling space in the healthcare facility for the medical procedure based on the further modified procedural time.

36. The method of claim 32 further comprising providing a wireless tracker module configured to move with at least one from the group consisting of the patient, a medical professional, hospital staff and a medical device to determine the time and location of the wireless tracker module within the healthcare environment.
